SEQUENCE LISTING (21 sequences)

SEQ ID NO: 1 - protein, 13 aa, synthetic construct
  source 1..13: note = Peptide 1
GADGAPGKDGVRG

SEQ ID NO: 2 - protein, 11 aa, synthetic construct
  source 1..11: note = Peptide 2
GPRGDQGPVGR

SEQ ID NO: 3 - protein, 9 aa, synthetic construct
  source 1..9: note = Peptide 3
GPTGPIGSR

SEQ ID NO: 4 - protein, 9 aa, synthetic construct
  source 1..9: note = Peptide 4
GPAGPAGRP

SEQ ID NO: 5 - protein, 4 aa, synthetic construct
  source 1..4: note = Peptide 5
GRYY

SEQ ID NO: 6 - protein, 10 aa, synthetic construct
  source 1..10: note = Peptide 6
GEGGPQGPRG

SEQ ID NO: 7 - protein, 12 aa, synthetic construct
  source 1..12: note = Peptide 7
GPRGDQGPVGRS

SEQ ID NO: 8 - protein, 4 aa, synthetic construct
  source 1..4: note = Peptide 8
FGFD

SEQ ID NO: 9 - protein, 4 aa, synthetic construct
  source 1..4: note = Peptide 9
FSGL

SEQ ID NO: 10 - protein, 9 aa, synthetic construct
  source 1..9: note = Peptide 10
GPAGPIGPV

SEQ ID NO: 11 - protein, 9 aa, synthetic construct
  source 1..9: note = Peptide 11
GPAGPQGPR

SEQ ID NO: 12 - protein, 7 aa, synthetic construct
  source 1..7: note = Peptide 12
LPQPPQE

SEQ ID NO: 13 - protein, 16 aa, synthetic construct
  source 1..16: note = Peptide 13
GPSGPAGKDGRIGQPG

SEQ ID NO: 14 - protein, 13 aa, synthetic construct
  source 1..13: note = Peptide 14
SGPAGPRGPPGSA

SEQ ID NO: 15 - protein, 6 aa, synthetic construct
  source 1..6: note = Peptide 15
GPVGPV

SEQ ID NO: 16 - protein, 21 aa, synthetic construct
  source 1..21: note = Peptide 16
GKSGDRGETGPAGPAGPIGPV

SEQ ID NO: 17 - protein, 4 aa, synthetic construct
  source 1..4: note = Peptide 17
PGEK

SEQ ID NO: 18 - protein, 4 aa, synthetic construct
  source 1..4: note = Peptide 18
SPDF

SEQ ID NO: 19 - protein, 13 aa, synthetic construct
  source 1..13: note = Peptide 19
GPAGKDGRIGQPG

SEQ ID NO: 20 - protein, 4 aa, synthetic construct
  source 1..4: note = Peptide 20
GERG

SEQ ID NO: 21 - protein, 7 aa, synthetic construct
  source 1..7: note = Peptide 21
GFDGDFY

(19) United States
(12) Patent Application Publication
(10) Pub. No.: US 2025/0066453 A1
(43) Pub. Date: Feb. 27, 2025

(54) PREPARATION METHOD AND USE OF YAK HIDE-DERIVED OLIGOPEPTIDE FERROUS CHELATE WITH HIGH ANTIOXIDANT ACTIVITY

(71) Applicants: TIANJIN UNIVERSITY OF SCIENCE AND TECHNOLOGY, Tianjin (CN); TIANJIN JIANLI TECHNOLOGY R & D CO., LTD., Tianjin (CN)

(72) Inventors: Tao WU, Tianjin (CN); Xiaoman CI, Tianjin (CN); Yongde CHEN, Tianjin (CN); Yashi LI, Tianjin (CN); Mengfan REN, Tianjin (CN); Jinjin YIN, Tianjin (CN); Rui LIU, Tianjin (CN); Min ZHANG, Tianjin (CN); Wenjie SUI, Tianjin (CN)

(73) Assignees: TIANJIN UNIVERSITY OF SCIENCE AND TECHNOLOGY, Tianjin (CN); TIANJIN JIANLI TECHNOLOGY R & D CO., LTD., Tianjin (CN)

(21) Appl. No.: 18/647,098

(22) Filed: Apr. 26, 2024

(30) Foreign Application Priority Data
Aug. 25, 2023 (CN) 202311079587.5

(51) Int. Cl. C07K 14/78; C07K 1/12; C07K 1/36; C12N 9/64

(52) U.S. Cl. CPC C07K 14/78; C07K 1/12; C07K 1/36; C12N 9/641; C12Y 304/22002; C12Y 304/22004

(57) ABSTRACT

The present disclosure provides a preparation method of a yak hide-derived oligopeptide ferrous chelate with a high antioxidant activity. The preparation method includes preparing a yak hide-derived oligomeric collagen peptide and subjecting the yak hide-derived oligomeric collagen peptide as a protein source to chelation with an iron source in water. A pretreated yak hide is subjected to enzymatic hydrolysis under a pH value of 7 at 50° C. for 4 h with an amount of an enzyme added at 2% to obtain a yak skin-derived collagen with a molecular weight of less than 2 kDa; the chelation is conducted in a peptide-to-iron mass ratio of 1:1 to 5:1 with a peptide concentration of 1% to 5% at 30° C. to 70° C. for 20 min to 60 min under a pH value of 3 to 8; and an iron chelating capacity is 42.72 mg/g under optimal preparation conditions.

### CROSS REFERENCE TO RELATED APPLICATION

This patent application claims the benefit and priority of Chinese Patent Application No. 202311079587.5, filed with the China National Intellectual Property Administration on Aug. 25, 2023, the disclosure of which is incorporated by reference herein in its entirety as part of the present application.

### REFERENCE TO SEQUENCE LISTING

A computer readable XML file entitled “HLP20240100165”, created on Mar. 22, 2024, with a file size of about 22, 661 bytes, contains the sequence listing for this application, has been filed with this application, and is hereby incorporated by reference in its entirety.

### TECHNICAL FIELD

The present disclosure belongs to the technical field of functional polypeptide chelating ferrous ions, especially relates to a preparation method of a yak hide-derived oligopeptide ferrous chelate with a high antioxidant activity using a yak hide-derived oligopeptide and ferrous sulfate as raw materials, and use.

### BACKGROUND

Iron is one of the essential micronutrients for most living organisms. The iron has a variety of biological functions, such as oxygen transport, DNA synthesis, and cell growth. However, iron deficiency anemia (IDA) caused by iron deficiency has become one of the most common nutritional deficiencies in humans, affecting approximately 2 billion people, especially pregnant women and children. Importantly, dysregulation of iron homeostasis in the human brain increases a risk of neurodegenerative diseases, including Alzheimer's disease, Parkinson's disease, and amyotrophic lateral sclerosis. Insufficient iron intake and low iron utilization are the main causes of iron deficiency. In addition, iron absorption inhibitors and promoters present in the diet can also affect a bioavailability of iron in the human body. A diet rich in protein, B-complex vitamins, and ascorbic acid promotes iron storage, while phytic acid, polyphenols, oxalic acid, and fiber hinder iron absorption. Currently, the intake of iron-fortified foods has become an effective approach to prevent and improve IDA globally.

Traditional iron supplements include ferrous sulfate, ferrous chloride, and ferrous lactate. However, these iron supplements suffer from gastrointestinal irritation, low human bioavailability, and strong adverse reactions. As an amino acid chelator, ferrous glycinate has a bioavailability 2.5 to 3.4 times that of ferrous sulfate and shows minimal gastrointestinal symptoms. However, the ferrous glycinate has also been gradually eliminated due to defects such as high cost of amino acids, unnecessary color development reactions, and fat oxidation. Therefore, there is an urgent need for a novel iron supplement without side effects. Iron-chelating peptides have been developed and used as novel iron supplements and are reported to have higher bioavailability without any adverse effects. On one hand, iron-chelating peptides can improve the bioavailability of ferrous ions through polypeptide channels and promote multi-channel absorption of iron. On the other hand, the iron-chelating peptide can effectively avoid the adverse effects of phytic acid and fiber on the utilization of free iron ions through the presence of metal chelating peptide. The improvement of iron bioavailability in vivo caused by iron-chelating peptides has been confirmed by multiple studies. Furthermore, the unique physiological functions of a peptide may not disappear upon chelation with ferrous ions. Polypeptide-ferrous chelates not only improve iron bioavailability, but also carry the functional properties of the peptide. Accordingly, a peptide-ferrous chelate may exhibit dual nutritional effects of functional peptide activity and iron supplementation. In view of this, the development of peptide-ferrous chelates as a novel bionutrient is of great significance.

Yak is a species that grows in green, natural, pollution-free, and high-altitude areas. The yak can adapt to harsh ecological environments such as strong radiation, low temperatures, large temperature differences, and hypoxia, and has a unique biological activity and an extremely high nutritional value. The rich nutrients contained in a yak hide can be further processed to increase its added output values. One of the directions is to produce collagen and deep-processed products thereof using the yak hide as a raw material. A large number of studies have shown that the yak skin-derived collagen peptide has the effects of lowering blood pressure, lowering blood glucose, stopping bleeding, replenishing blood, antioxidant, and beautifying.

### SUMMARY

To overcome the deficiencies in the prior art, an objective of the present disclosure is to provide a preparation method and use of a yak hide-derived oligopeptide ferrous chelate with a high antioxidant activity.

The present disclosure resolves the technical problems with following technical solutions:

The present disclosure provides a preparation method of a yak hide-derived oligopeptide ferrous chelate with a high antioxidant activity, including subjecting a protein source and an iron source to chelation in water, where the protein source is a yak skin-derived collagen oligopeptide with a molecular weight of less than 2 kDa; and the yak skin-derived collagen oligopeptide and the iron source are subjected to the chelation in a mass ratio of 1:1 to 5:1 with a mass concentration of the yak skin-derived collagen oligopeptide of 1% to 5% under a pH value of 3 to 8 at 30° C. to 70° C. for 20 min to 60 min.

Further, the yak skin-derived collagen oligopeptide (protein source) and the iron source are subjected to the chelation in a mass ratio of 2.7:1 with a mass concentration of the yak skin-derived collagen oligopeptide of 3% under a pH value of 6.8 at 50° C. for 40 min; and a corresponding prepared yak hide-derived oligopeptide ferrous chelate shows an iron chelating capacity of 42.72 mg/g±0.65 mg/g.

Further, the yak skin-derived collagen oligopeptide is a peptide segment with a relatively high iron chelating capacity and includes: SEQ ID NO: 1: GADGAPGKDGVRG and SEQ ID NO: 2: GPRGDQGPVGR.

Further, the preparation method specifically includes the following steps:
(1) pretreating a yak hide: mechanically depilating the yak hide, removing subcutaneous muscle and fat, rinsing with water to remove surface soft flocks, cutting the yak hide into small pieces of 1 cm×1 cm, and subjecting the yak hide to defatting with a 5% Na₂CO₃ aqueous solution at 4° C. for 18 h to obtain a defatted yak hide; adding a NaCl solution with a mass fraction of 5% into the defatted yak hide and stirring continuously to remove salt-soluble non-collagen components, where the defatted yak hide and the NaCl solution are at a material-to-liquid ratio of 1:10, rinsing the defatted yak hide with distilled water multiple times to obtain a clean yak skin, and storing the clean yak hide at −20° C. for later use; adding the clean yak skin into 0.5 mol/L glacial acetic acid at a material-to-liquid ratio of 1:10 to allow swelling for 12 h, and conducting homogenization using a high-speed tissue masher at 10,000 r/min to obtain a yak hide homogenate to allow subsequent enzymatic hydrolysis;
(2) conducting enzymatic hydrolysis: subjecting the yak hide homogenate to enzymatic hydrolysis using different proteases of neutral protease, alkaline protease, flavor protease, complex protease, papain, and bromelain separately; where the enzymatic hydrolysis includes: the enzymatic hydrolysis using the alkaline protease is conducted at a pH value of 10, while the enzymatic hydrolysis using the other proteases is conducted at a pH value of 7, and the enzymatic hydrolysis is conducted at 50° C. for 4 h with an amount of the protease added at 2% of a mass of the yak hide homogenate; heating a resulting enzymatic hydrolyzate at 95° C. for 15 min to terminate the enzymatic hydrolysis, and then conducting centrifugation under a room temperature at 5,000 r/min for 20 min to obtain a supernatant; precipitating a polysaccharide in the supernatant using absolute ethanol, and then conducting centrifugation at 4,000 r/min for 20 min to obtain a yak skin-derived collagen peptide solution obtained by the enzymatic hydrolysis using each of the different proteases;
(3) conducting separation and purification by gel chromatography: subjecting the yak skin-derived collagen peptide solution obtained by the enzymatic hydrolysis using each of the different proteases to separation and purification by gel chromatography to obtain a yak skin-derived collagen peptide; subjecting the yak skin-derived collagen peptide to subsequent enzymatic hydrolysis using the flavor protease under a pH value of 7 at 50° C. for 4 h with an amount of the flavor protease added at 2% of a mass of the yak skin-derived collagen peptide to obtain the yak hide-derived collagen oligopeptide;
(4) mixing the yak skin-derived collagen oligopeptide and a FeSO₄·7H₂O solution with a mass concentration of 1% to 5% to obtain a mixed solution, where the yak skin-derived collagen oligopeptide and the FeSO₄·7H₂O solution are at a mass-to-volume ratio of (0.1-0.5) g:10 mL, adjusting the mixed solution to a pH value of 3 to 8 with 1 mol/L NaOH or 1 mol/L HCl, and subjecting the mixed solution to the chelation at 30° C. to 70° C. for 20 min to 60 min; and
(5) adding 4 times a volume of the absolute ethanol into a resulting reaction product to precipitate a chelate of a yak hide-derived oligopeptide and ferrous ions after the chelation is completed; conducting centrifugation at 10,000 r/min for 15 min, and collecting a resulting precipitate to allow freeze-drying to obtain the yak hide-derived oligopeptide ferrous chelate with a high antioxidant activity.

Further, a yak hide-derived oligopeptide ferrous chelate prepared by the preparation method has a better capacity in scavenging a 2,2-diphenyl-1-picrylhydrazyl (DPPH) free radical and a 2,2′-azino-bis(3-ethylbenzothiazoline-6-sulfonic acid) (ABTS) free radical than that of a yak hide-derived oligomeric collagen peptide.

Further, the yak hide-derived oligopeptide ferrous chelate includes 11.18%±1.08% of iron.

The present disclosure provides use of a yak hide-derived oligopeptide ferrous chelate prepared by the preparation method in production of a food and/or a drug and/or a health care product.

The present disclosure has the following advantages and beneficial effects:

1. In the present disclosure, a yak hide-derived iron-chelating peptide is obtained by studying reaction conditions such as peptide-to-iron mass ratio, peptide concentration, reaction time, reaction temperature, and solution pH, and shows high iron content and antioxidant activity. The yak hide-derived iron-chelating peptide serves as a novel functional additive with multiple nutritional functions such as iron supplementation, antioxidant, and amino acid supplementation, and can be used for preparing food, drugs, and health care products.

2. The present disclosure aims to obtain a yak hide-derived oligomeric collagen peptide through enzymatic hydrolysis of a yak hide, and then organically combine the yak hide-derived oligomeric collagen peptide with ferrous sulfate to prepare a novel iron-chelating peptide. A peptide sequence of the yak hide-derived oligopeptide ferrous chelate is identified through liquid chromatography-mass spectrometry (LC-MS), and two peptide segments (GADGAPGKDGVRG (SEQ ID NO: 1) and GPRGDQGPVGR (SEQ ID NO: 2)) with a relatively high iron chelating capacity are selected through molecular docking. In addition, an in vitro antioxidant effect of the iron-chelating peptide is evaluated by a scavenging capacity of 2,2-diphenyl-1-picrylhydrazyl (DPPH) free radical and a 2,2′-azino-bis(3-ethylbenzothiazoline-6-sulfonic acid) (ABTS) free radical, providing a more comprehensive theoretical basis for the development of iron-fortified food industry.

3. In the present disclosure, a protein source when preparing the yak hide-derived iron-chelating peptide is a yak hide-derived oligomeric collagen peptide; and the yak hide-derived oligomeric collagen peptide and the iron source are subjected to the chelation in a mass ratio of 1:1 to 5:1 under a pH value of 3 to 8 at 30° C. to 70° C. for 20 min to 60 min. After single factor and response surface optimization, the optimal conditions for preparing yak hide-derived oligopeptide ferrous chelate are obtained. A ferrous chelate prepared under these conditions has an iron content of 42.72 mg/g±0.65 mg/g. The present disclosure further discloses two peptide segments with a relatively high iron chelating activity: GADGAPGKDGVRG (SEQ ID NO: 1) and GPRGDQGPVGR (SEQ ID NO: 2). The binding site and binding energy of a peptide chain and ferrous ions are predicted through molecular docking. The results show that the GADGAPGKDGVRG (SEQ ID NO: 1) and GPRGDQGPVGR (SEQ ID NO: 2) each have binding energy of 4.17 KJ/mol to the ferrous ions, indicating relatively high binding energy, which further provides effective evidence for the development of iron-chelating peptides. The yak hide-derived iron-chelating peptide has multiple biological effects such as antioxidant, iron supplementation, and amino acid supplementation, and provides new ideas for the high-value utilization of yak hide and the development of novel iron-fortified foods.

4. In the present disclosure, it is found that the chelating sites for the combination of yak hide-derived oligopeptide and ferrous ions are amino groups, carboxyl groups, and carbonyl groups, and a binding method is mainly electrostatic interaction. In addition, the yak skin-derived oligomeric collagen peptide can be used to prepare iron-chelating peptides.

### BRIEF DESCRIPTION OF THE DRAWINGS

FIGS. 1A, 1B show molecular weight distribution and gel chromatography of yak hide-derived collagen peptides obtained by enzymatic hydrolysis with different proteases in the present disclosure;

FIG. 2 shows the Fourier transform infrared absorption spectrums of yak hide-derived oligomeric collagen peptide and yak hide-derived oligomeric collagen peptide ferrous chelate in the present disclosure; where YSP represents the yak hide-derived oligomeric collagen peptide, and YSP-Fe represents the yak hide-derived oligomeric collagen peptide ferrous chelate;

FIGS. 3A, 3B, 3C, 3D, 3E, 3F show the microstructure of the yak hide-derived oligomeric collagen peptide and the yak hide-derived oligomeric collagen peptide ferrous chelate in the present disclosure; where FIG. 3A, FIG. 3B, and FIG. 3C are the microstructures of the yak hide-derived oligomeric collagen peptide observed at magnifications of 1,000, 3,000, and 5,000; and FIG. 3D, FIG. 3E, and FIG. 3F are the microstructures of the yak hide-derived oligomeric collagen peptide ferrous chelate observed at magnifications of 1,000, 3,000, and 5,000;

FIGS. 4A, 4B show the energy spectrums of yak hide-derived oligomeric collagen peptide and yak hide-derived oligomeric collagen peptide ferrous chelate in the present disclosure; where YSP represents the yak hide-derived oligomeric collagen peptide, and YSP-Fe represents the yak hide-derived oligomeric collagen peptide ferrous chelate;

FIGS. 5A, 5B show the molecular docking results of GADGAPGKDGVRG (SEQ ID NO: 1) and GPRGDQGPVGR (SEQ ID NO: 2) with ferrous ions in the present disclosure; where FIG. 5A is a molecular docking model of the GADGAPGKDGVRG (SEQ ID NO: 1) and ferrous ions; FIG. 5B is a molecular docking model of the GPRGDQGPVGR (SEQ ID NO: 2) and ferrous ions; notes: red represents oxygen atoms, blue represents nitrogen atoms, and white represents hydrogen atoms; and

FIGS. 6A, 6B show the antioxidant activity analysis results of the yak hide-derived oligopeptide ferrous chelate in the present disclosure; where YSP represents the yak hide-derived oligomeric collagen peptide, and YSP-Fe represents the yak hide-derived oligomeric collagen peptide ferrous chelate.

### DETAILED DESCRIPTION OF THE EMBODIMENTS

The disclosure is further illustrated with reference to the following examples which are illustrative and not restrictive. The scope of protection of the disclosure is not limited by the following examples.

Various experimental operations involved in the specific examples are routine techniques in this field. If there are no special annotations in this article, those of ordinary skill in the art can refer to various commonly used reference books, scientific and technological documents or related instructions and manuals before the filing date of the present disclosure for implementation.

The present disclosure provides a preparation method of a yak hide-derived oligopeptide ferrous chelate with a high antioxidant activity, including subjecting a protein source and an iron source to chelation in water, where the protein source is a yak skin-derived collagen oligopeptide with a molecular weight of less than 2 kDa; and the yak skin-derived collagen oligopeptide and the iron source are subjected to the chelation in a mass ratio of 1:1 to 5:1 with a mass concentration of the yak skin-derived collagen oligopeptide of 1% to 5% under a pH value of 3 to 8 at 30° C. to 70° C. for 20 min to 60 min.

Preferably, the yak skin-derived collagen oligopeptide (protein source) and the iron source are subjected to the chelation in a mass ratio of 2.7:1 with a mass concentration of the yak skin-derived collagen oligopeptide of 3% under a pH value of 6.8 at 50° C. for 40 min; and a corresponding prepared yak hide-derived oligopeptide ferrous chelate shows an iron chelating capacity of 42.72 mg/g±0.65 mg/g.

Preferably, the yak skin-derived collagen oligopeptide is a peptide segment with a relatively high iron chelating capacity and includes SEQ ID NO: 1: GADGAPGKDGVRG and SEQ ID NO: 2: GPRGDQGPVGR.

Preferably, the preparation method specifically includes the following steps:
(1) pretreating a yak hide: mechanically depilating the yak hide, removing subcutaneous muscle and fat, rinsing with water to remove surface soft flocks, cutting the yak hide into small pieces of 1 cm×1 cm, and subjecting the yak hide to defatting with a 5% Na₂CO₃ aqueous solution at 4° C. for 18 h to obtain a defatted yak hide; adding a NaCl solution with a mass fraction of 5% into the defatted yak hide and stirring continuously to remove salt-soluble non-collagen components, where the defatted yak hide and the NaCl solution are at a material-to-liquid ratio of 1:10, rinsing the defatted yak hide with distilled water multiple times to obtain a clean yak skin, and storing the clean yak hide at −20° C. for later use; adding the clean yak skin into 0.5 mol/L glacial acetic acid at a material-to-liquid ratio of 1:10 to allow swelling for 12 h, and conducting homogenization using a high-speed tissue masher at 10,000 r/min to obtain a yak hide homogenate to allow subsequent enzymatic hydrolysis;
(2) conducting enzymatic hydrolysis: subjecting the yak hide homogenate to enzymatic hydrolysis using different proteases of neutral protease, alkaline protease, flavor protease, complex protease, papain, and bromelain separately; where the enzymatic hydrolysis includes: the enzymatic hydrolysis using the alkaline protease is conducted at a pH value of 10, while the enzymatic hydrolysis using the other proteases is conducted at a pH value of 7, and the enzymatic hydrolysis is conducted at 50° C. for 4 h with an amount of the protease added at 2% of a mass of the yak hide homogenate; heating a resulting enzymatic hydrolyzate at 95° C. for 15 min to terminate the enzymatic hydrolysis, and then conducting centrifugation under a room temperature at 5,000 r/min for 20 min to obtain a supernatant; precipitating a polysaccharide in the supernatant using absolute ethanol, and then conducting centrifugation at 4,000 r/min for 20 min to obtain a yak skin-derived collagen peptide solution obtained by the enzymatic hydrolysis using each of the different proteases;
(3) conducting separation and purification by gel chromatography: subjecting the yak skin-derived collagen peptide solution obtained by the enzymatic hydrolysis using each of the different proteases to separation and purification by gel chromatography to obtain a yak skin-derived collagen peptide; subjecting the yak skin-derived collagen peptide to subsequent enzymatic hydrolysis using the flavor protease under a pH value of 7 at 50° C. for 4 h with an amount of the flavor protease added at 2% of a mass of the yak skin-derived collagen peptide to obtain the yak hide-derived collagen oligopeptide;
(4) mixing the yak skin-derived collagen oligopeptide and a FeSO₄·7H₂O solution with a mass concentration of 1% to 5% to obtain a mixed solution, where the yak skin-derived collagen oligopeptide and the FeSO₄·7H₂O solution are at a mass-to-volume ratio of (0.1-0.5) g:10 mL, adjusting the mixed solution to a pH value of 3 to 8 with 1 mol/L NaOH or 1 mol/L HCl, and subjecting the mixed solution to the chelation at 30° C. to 70° C. for 20 min to 60 min; and
(5) adding 4 times a volume of the absolute ethanol into a resulting reaction product to precipitate a chelate of a yak hide-derived oligopeptide and ferrous ions after the chelation is completed; conducting centrifugation at 10,000 r/min for 15 min, and collecting a resulting precipitate to allow freeze-drying to obtain the yak hide-derived oligopeptide ferrous chelate with a high antioxidant activity.

Preferably, a yak hide-derived oligopeptide ferrous chelate prepared by the preparation method has a better capacity in scavenging a DPPH free radical and an ABTS free radical than that of a yak hide-derived oligomeric collagen peptide.

Preferably, the yak hide-derived oligopeptide ferrous chelate includes 11.18%=1.08% of iron.

The present disclosure provides use of a yak hide-derived oligopeptide ferrous chelate prepared by the preparation method in production of a food and/or a drug and/or a health care product.

Specifically, the relevant preparation and detection are as follows:

In the present disclosure, a yak hide-derived oligomeric collagen peptide with a molecular weight of less than 2 kDa is obtained through separation and purification by gel chromatography, and then chelated with ferrous ions to obtain a yak hide-derived oligomeric collagen peptide ferrous chelate. In addition, the binding models of the two peptide segments GADGAPGKDGVRG (SEQ ID NO: 1) and GPRGDQGPVGR (SEQ ID NO: 2) with ferrous ions are simulated through molecular docking and their binding sites are also predicted.

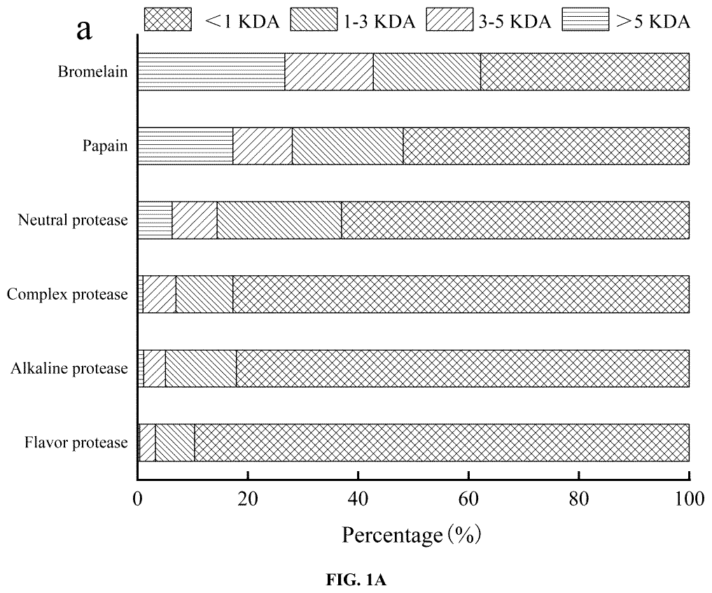

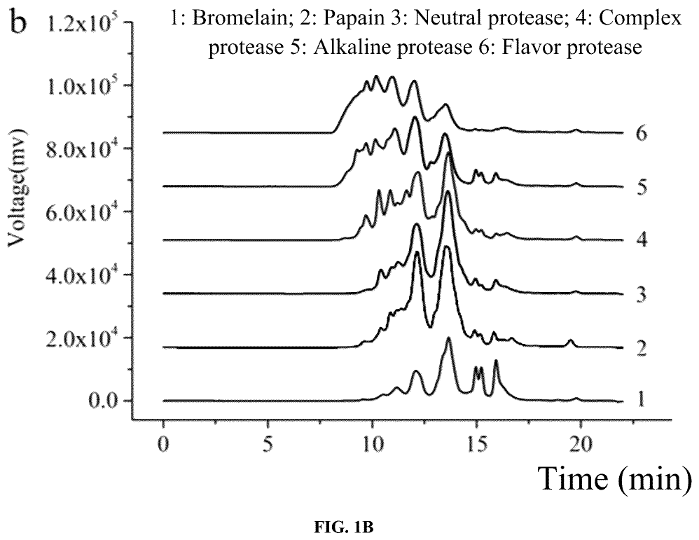

Example 1: A preparation method of a yak hide-derived oligopeptide ferrous chelate with a high antioxidant activity included the following steps:
(1) pretreating a yak hide: the yak hide was mechanically depilated, subcutaneous muscle and fat were removed, the yak hide was rinsed with water to remove surface soft flocks, cut into small pieces of 1 cm×1 cm, and then subjected to defatting with a 5% Na₂CO₃ aqueous solution at 4° C. for 18 h to obtain a defatted yak hide; a NaCl solution with a mass fraction of 5% was added into the defatted yak hide and stirred continuously to remove salt-soluble non-collagen components, where the defatted yak hide and the NaCl solution were at a material-to-liquid ratio of 1:10, the defatted yak hide was rinsed with distilled water multiple times to obtain a clean yak skin, and the clean yak hide was stored at −20° C. for later use; the clean yak skin was added into 0.5 mol/L glacial acetic acid at a material-to-liquid ratio of 1:10 to allow swelling for 12 h, and homogenization was conducted using a high-speed tissue masher at 10,000 r/min to obtain a yak hide homogenate to allow subsequent enzymatic hydrolysis;
(2) conducting enzymatic hydrolysis: the yak hide homogenate was subjected to enzymatic hydrolysis using different proteases of neutral protease, alkaline protease, flavor protease, complex protease, papain, and bromelain separately; where the enzymatic hydrolysis included: the enzymatic hydrolysis using the alkaline protease was conducted at a pH value of 10, while the enzymatic hydrolysis using the other proteases was conducted at a pH value of 7, and the enzymatic hydrolysis was conducted at 50° C. for 4 h with an amount of the protease added at 2% of a mass of the yak hide homogenate; a resulting enzymatic hydrolyzate was heated at 95° C. for 15 min to terminate the enzymatic hydrolysis, and then centrifugation was conducted under a room temperature at 5,000 r/min for 20 min to obtain a supernatant; a polysaccharide in the supernatant was precipitated using absolute ethanol, and then centrifugation was conducted at 4,000 r/min for 20 min to obtain a yak skin-derived collagen peptide solution obtained by the enzymatic hydrolysis using each of the different proteases;
(3) conducting separation and purification by gel chromatography: the yak skin-derived collagen peptide solution obtained by the enzymatic hydrolysis using each of the different proteases was subjected to separation and purification by gel chromatography and molecular weight distribution determination; as shown in FIGS. 1A-1B, the largest proportion of yak hide-derived collagen peptide obtained by enzymatic hydrolysis of flavor protease had a molecular weight of less than 1,000 Da, such that the flavor protease was selected for subsequent enzymatic hydrolysis of yak hide-derived collagen peptide; the yak skin-derived collagen peptide was subjected to enzymatic hydrolysis using the flavor protease under a pH value of 7 at 50° C. for 4 h with an amount of the flavor protease added at 2% of a mass of the yak skin-derived collagen peptide to obtain the yak hide-derived collagen oligopeptide;
(4) the yak skin-derived collagen oligopeptide (0.1 g to 0.5 g) and 10 mL of a FeSO₄·7H₂O solution with a mass concentration of 1% to 5% (w/w) were mixed to obtain a mixed solution, the mixed solution was adjusted to a pH value of 3 to 8 with 1 mol/L NaOH or 1 mol/L HCl, and the mixed solution was subjected to the chelation at 30° C. to 70° C. for 20 min to 60 min; and
(5) the absolute ethanol (1:4 by volume ratio, v/v) was added into a resulting reaction product to precipitate a chelate of a yak hide-derived oligopeptide and ferrous ions after the chelation was completed; centrifugation was conducted at 10,000 r/min for 15 min, and a resulting precipitate was collected to allow freeze-drying to obtain the yak hide-derived oligopeptide ferrous chelate with a high antioxidant activity.

Example 2: In the preparation method of a yak hide-derived oligopeptide ferrous chelate with a high antioxidant activity, the steps were the same as those in Example 1, except that: 0.3 g of a yak hide-derived collagen oligopeptide was mixed with 10 mL of a 3% FeSO₄·7H₂O solution to obtain a mixed solution, and the mixed solution was adjusted to a pH value of to 7 with 1 mol/L NaOH or 1 mol/L HCl. The chelation was conducted at 50° C. for 40 min.

Example 3: In the preparation method of a yak hide-derived oligopeptide ferrous chelate with a high antioxidant activity, the steps were the same as those in Example 1, except that: 0.4 g of a yak hide-derived collagen oligopeptide was mixed with 10 mL of a 4% FeSO₄·7H₂O solution to obtain a mixed solution, and the mixed solution was adjusted to a pH value of to 8 with 1 mol/L NaOH or 1 mol/L HCl. The chelation was conducted at 55° C. for 45 min.

Example 4: In the preparation method of a yak hide-derived oligopeptide ferrous chelate with a high antioxidant activity, the steps were the same as those in Example 1, except that: 0.2 g of a yak hide-derived collagen oligopeptide was mixed with 10 mL of a 2% FeSO₄·7H₂O solution to obtain a mixed solution, and the mixed solution was adjusted to a pH value of to 6 with 1 mol/L NaOH or 1 mol/L HCl. The chelation was conducted at 45° C. for 35 min.

Comparative Example 1: In the preparation method of a yak hide-derived oligopeptide ferrous chelate, the steps were the same as those in Example 2, except that: 0.3 g of yak hide-derived oligomeric collagen peptide was mixed with 10 mL of a 3% FeSO₄·7H₂O solution to obtain a mixed solution, and the mixed solution was adjusted to a pH value of to 10 with 1 mol/L NaOH or 1 mol/L HCl. The chelation was conducted at 50° C. for 40 min.

Comparative Example 2: In the preparation method of a yak hide-derived oligopeptide ferrous chelate, the steps were the same as those in Example 2, except that: 0.3 g of yak hide-derived oligomeric collagen peptide was mixed with 10 mL of a 3% FeSO₄·7H₂O solution to obtain a mixed solution, and the mixed solution was adjusted to a pH value of to 2 with 1 mol/L NaOH or 1 mol/L HCl. The chelation was conducted at 50° C. for 40 min.

Comparative Example 3: In the preparation method of a yak hide-derived oligopeptide ferrous chelate, the steps were the same as those in Example 3, except that: 0.4 g of yak hide-derived oligomeric collagen peptide was mixed with 10 mL of a 0.5% FeSO₄·7H₂O solution to obtain a mixed solution, and the mixed solution was adjusted to a pH value of to 8 with 1 mol/L NaOH or 1 mol/L HCl. The chelation was conducted at 90° C. for 45 min.

Comparative Example 4: In the preparation method of a yak hide-derived oligopeptide ferrous chelate, the steps were the same as those in Example 3, except that: 0.4 g of yak hide-derived oligomeric collagen peptide was mixed with 10 mL of a 0.5% FeSO₄·7H₂O solution to obtain a mixed solution, and the mixed solution was adjusted to a pH value of to 8 with 1 mol/L NaOH or 1 mol/L HCl. The chelation was conducted at 55° C. for 45 min.

TABLE 1
Comparison of iron chelating capacity

| | Iron chelating capacity (mg/g) |
|---|---|
| Example 2 | 41.85 |
| Example 3 | 36.82 |
| Example 4 | 37.32 |
| Comparative Example 1 | 9.32 |
| Comparative Example 2 | 8.18 |
| Comparative Example 3 | 5.78 |
| Comparative Example 4 | 6.54 |

As shown in Table 1, the iron chelating capacity of the yak hide-derived oligopeptide ferrous chelate prepared in the present disclosure was significantly higher than that of the yak hide-derived oligopeptide ferrous chelate prepared under the chelating conditions of the comparative examples.

As shown in Example 2, Comparative Example 1, and Comparative Example 2, there was a synergistic effect between “adjusting the pH value of the mixed solution to 3 to 8 using 1 mol/L NaOH or 1 mol/L HCl” in the method of the present disclosure and other conditions. If the pH value was not within the range of “3 to 8”, the prepared yak hide-derived oligopeptide ferrous chelate might have a poor performance. The “adjusting the pH value of the mixed solution to 3 to 8 using 1 mol/L NaOH or 1 mol/L HCl” in the method of the present disclosure and other conditions could synergistically improve the relevant properties of the prepared yak hide-derived oligopeptide ferrous chelate.

As shown in Example 3, Comparative Example 3, and Comparative Example 4, there was a synergistic effect between the “ratio of yak hide-derived collagen oligopeptide: FeSO₄·7H₂O solution” and “chetation at 30° C. to 70° C.” in the method of the present disclosure. If the above two ranges were not within this range, the prepared yak hide-derived oligopeptide ferrous chelate might have a poor performance. The “ratio of yak hide-derived collagen oligopeptide: FeSO₄·7H₂O solution” and “chetation at 30° C. to 70° C.” in the method of the present disclosure could synergistically improve the relevant properties of the prepared yak hide-derived oligopeptide ferrous chelate.

### Example 5: Structural Characterization of Yak Hide-Derived Oligopeptide Ferrous Chelate

In order to confirm the formation of the yak hide-derived iron-chelating peptide and provide a research basis for the structure-activity relationship between the peptide and iron, the prepared peptide-iron chelate was characterized by infrared spectrum and microstructure.

### 1. Infrared Spectral Analysis

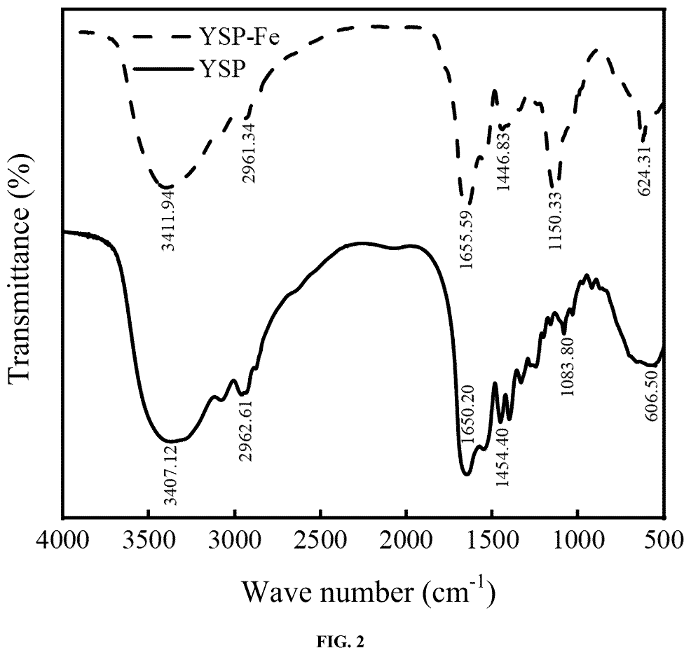

The change in an absorption peak of the infrared spectrum can reflect the interaction between metal ions and the organic groups of the peptide. The type of active groups in the chelation between yak hide-derived oligopeptide and ferrous ions could be determined through the wavelength shift of an infrared absorption peak of the characteristic group. As shown in FIG. 2, there are obvious differences in the absorption peaks of yak hide-derived oligopeptide and yak hide-derived oligopeptide ferrous chelate. Compared with the spectrum of yak hide-derived oligopeptide, the characteristic wave number of the amino groups in the yak hide-derived oligopeptide ferrous chelate moved from 3407.12 cm⁻¹ to 3411.94 cm⁻¹. This might be due to the participation of the N-terminal amino groups and the amino groups on an amino acid side chain in the chelation, resulting in a change in a vibration frequency of the N—H bond. An absorption band of yak hide-derived oligopeptide at 1650.20 cm⁻¹ was characterized as an amide I band, which was attributed to the coupling between stretching vibration of C=0 with the bending vibration of N—H. After combining with ferrous ions, a band corresponding to the amide I group moved to 1655.59 cm⁻¹, indicating that the C=0 group might combine with ferrous ions to generate C—O—Fe. When the yak hide-derived oligopeptide was chelated with ferrous ions, the absorption peak of —COOH moved from 1454.40 cm⁻¹ to 1446.83 cm⁻¹. This indicated that-COOH at the C-terminal (such as glutamic acid and aspartic acid residues) also participated in the chelation, and ferrous ions might replace H in —COOH to form —COO—Fe. In addition, 606.50 cm⁻¹ represented the characteristic wave number of the imidazole group. When the yak hide-derived oligopeptide was chelated, its characteristic wave number moved to 624.31 cm⁻¹, and it was speculated that N atoms in the imidazole group could combine with ferrous ions to form N—Fe.

It was seen that the binding sites for ferrous ions in yak hide-derived oligopeptide were mainly characteristic functional groups such as carboxyl, amino, carbonyl, and imidazole groups in the organic matter. The amino and imidazole groups contained lone pairs of electrons from N atoms, while the carboxyl group contained lone pairs of electrons from two O atoms. The lone pairs of electrons of the N and O atoms were combined with iron ions in the form of coordination bonds to form a chelate with a cyclic structure.

### 2. Microstructure Observation

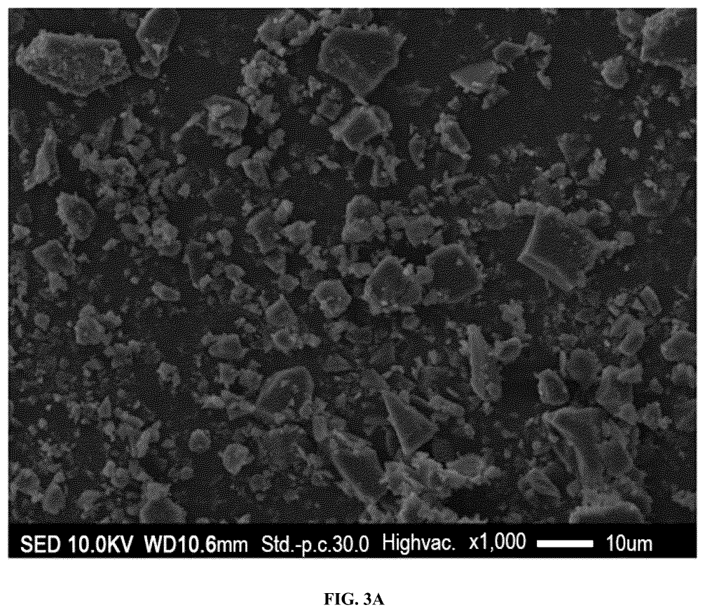

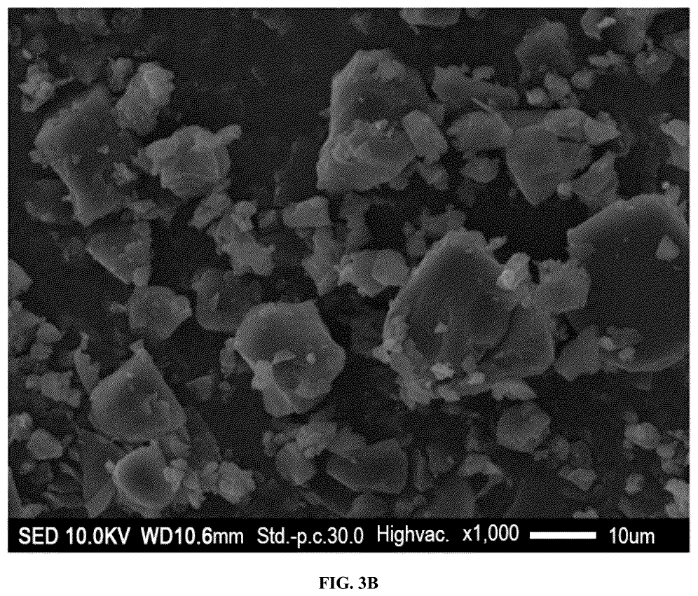

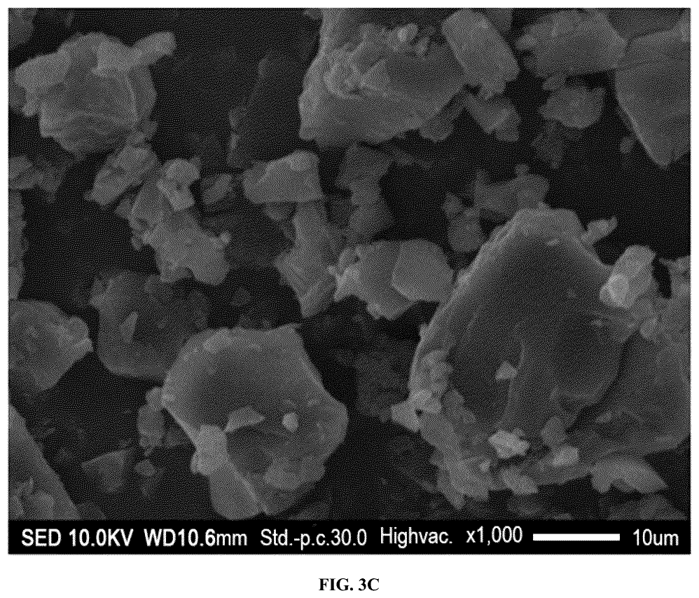

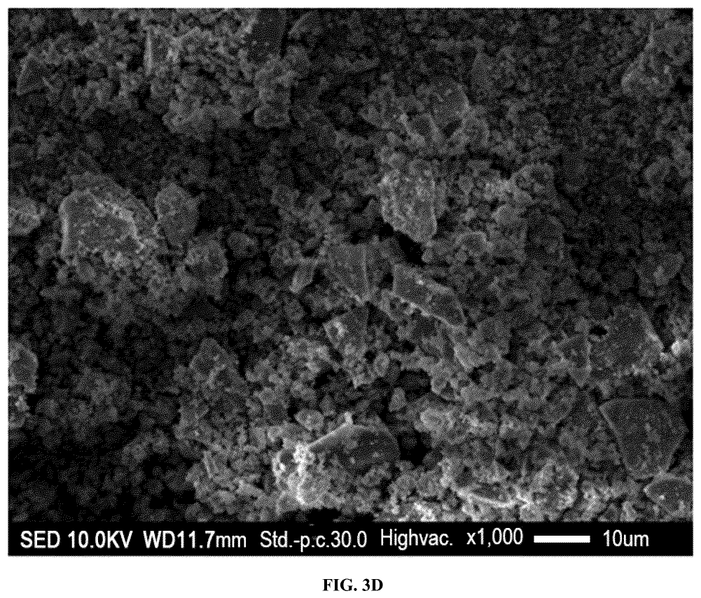

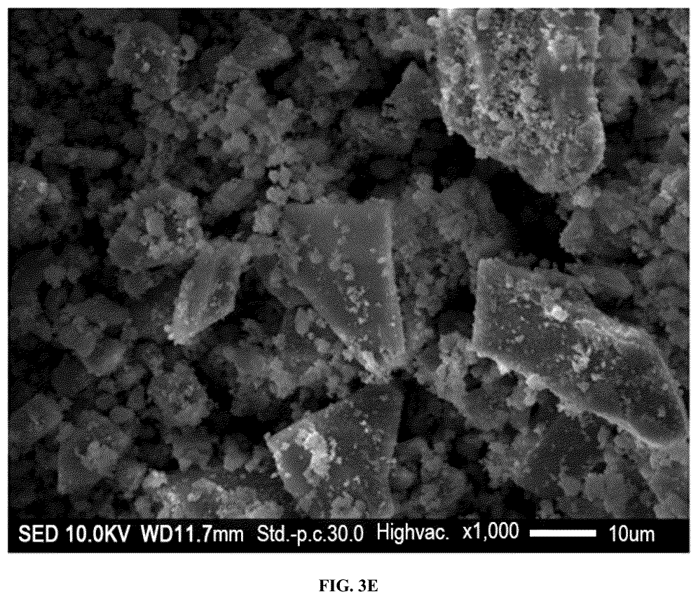

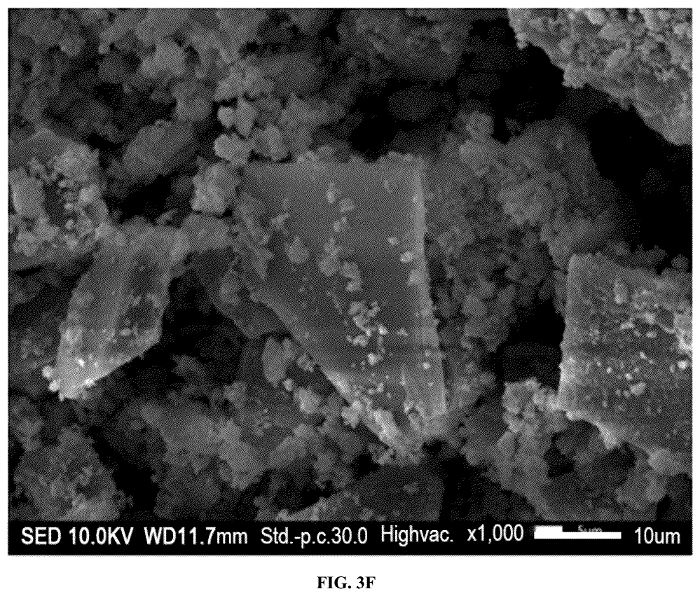

The morphological differences between yak hide-derived oligopeptide and yak hide-derived oligopeptide ferrous chelate were understood using scanning electron microscopy (SEM). As shown in FIGS. 3A-3F, the microstructure of the yak hide-derived oligopeptide and yak hide-derived oligopeptide ferrous chelate was magnified 1,000 times, 3,000 times, and 5,000 times separately. FIGS. 3A-3F showed that the surface of the yak hide-derived oligopeptide was smooth and dense, showing a block structure; while the surface of the yak hide-derived oligopeptide ferrous chelate was rough and loose, and there were crystals adsorbed on the surface of the chelated peptide. This might be that the iron crystals were adsorbed on the surface of the peptide after the reaction between the peptide and the iron salt. The changes in the microstructure during the formation of yak hide-derived oligopeptide ferrous chelates might be due to the combination of yak hide-derived oligopeptide and ferrous ions destroying the original smooth and dense structure of the peptide surface.

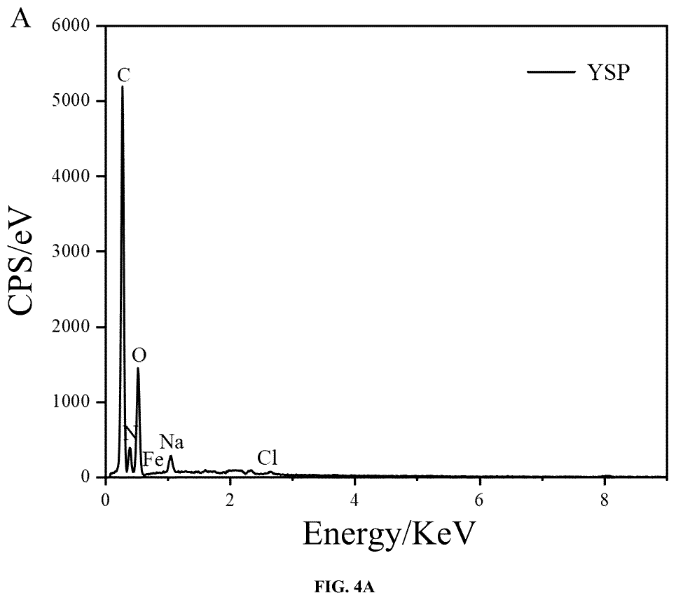

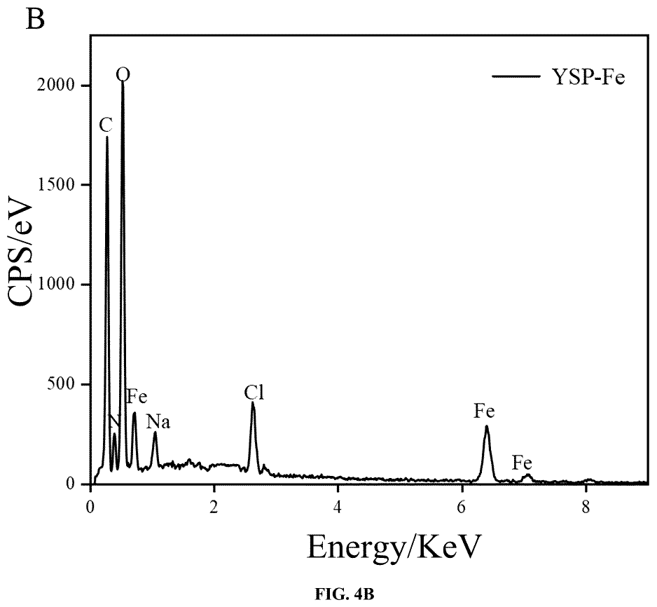

Energy dispersive spectrometry (EDS) is an effective method for analyzing the elemental composition of sample surfaces. As shown in FIGS. 4A-4B, there was an iron peak in the yak hide-derived oligopeptide with an extremely low content, while there were three iron peaks in the energy spectrum of the yak hide-derived oligopeptide ferrous chelate. The content of each element of yak hide-derived oligopeptide and yak hide-derived oligopeptide ferrous chelate was analyzed through the energy spectrum, as shown in Table 2. Yak hide-derived oligopeptide had 0.02%±0.02% of iron, while yak hide-derived oligopeptide ferrous chelate had 11.18%±1.08% of iron. The signal intensity and iron content of iron in YSP-Fe increased significantly, indicating that the yak hide-derived oligopeptide and ferrous ions were successfully chelated during the chelation.

TABLE 2
Elemental composition analysis of yak hide-derived oligopeptide
and yak hide-derived oligopeptide ferrous chelate

| | Element | | | | | |
|---|---|---|---|---|---|---|
| Sample | C (%) | N (%) | O (%) | Na (%) | Cl (%) | Fe (%) |
| YSP | 46.40 ± 0.87 | 20.41 ± 1.62 | 31.69 ± 2.36 | 1.19 ± 0.07 | 0.28 ± 0.10 | 0.02 ± 0.02 |
| YSP-Fe | 31.33 ± 1.52 | 10.13 ± 0.67 | 41.64 ± 3.12 | 2.50 ± 1.07 | 2.53 ± 0.84 | 11.86 ± 1.08 |

### Example 6: Molecular Model Establishment of GADGAPGKDGVRG (SEQ ID NO: 1) and GPRGDQGPVGR (SEQ ID NO: 2)

A peptide sequence of the yak hide-derived oligopeptide ferrous chelate was identified through LC-MS (Table 3), and two peptide segments (GADGAPGKDGVRG (SEQ ID NO: 1) and GPRGDQGPVGR (SEQ ID NO: 2)) with a relatively high iron chelating capacity were selected through molecular docking.

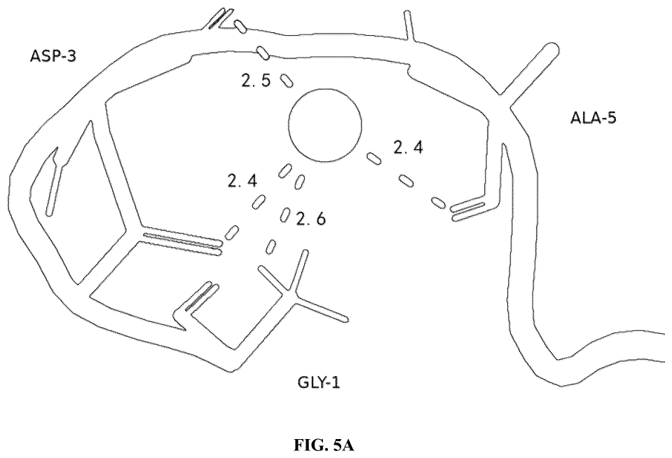

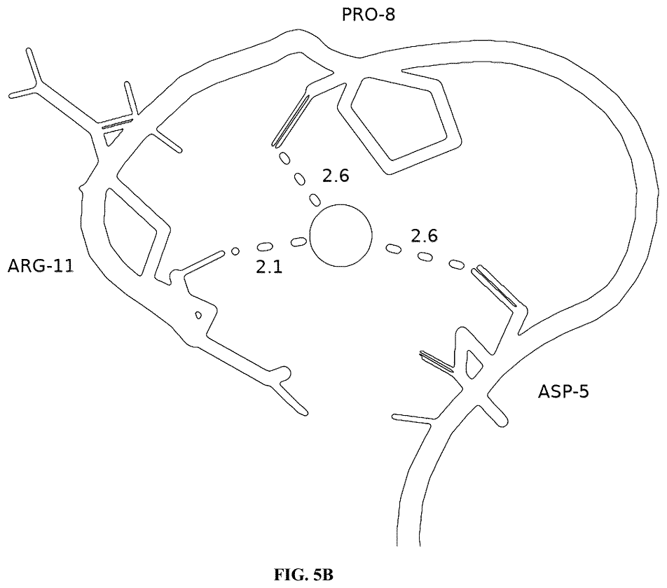

The binding models of the two peptide chains GADGAPGKDGVRG (SEQ ID NO: 1) and GPRGDQGPVGR (SEQ ID NO: 2) with ferrous ions were established through molecular docking and their binding sites were also predicted. As shown in FIG. 5A, the binding sites of GADGAPGKDGVRG (SEQ ID NO: 1) and ferrous ions were Asp3, Ala5, and Gly1, with binding energy of −4.17 KJ/mol. The results of the docking model showed that: there were two carboxyl groups in Asp3 that bound to ferrous ions, and the distances between the two carboxyl groups and the ferrous ions were 2.4 Ä and 2.5 Ä, respectively; one carboxyl group in Ala5 was bound to ferrous ions at a distance of 2.4 Ä; one amino group in Gly1 was bound to the ferrous ion at a distance of 2.6 Ä.

As shown in FIG. 5B, the binding sites of GPRGDQGPVGR (SEQ ID NO: 2) and ferrous ions were Asp5, Arg11, and Pro8, with binding energy of −4.17 KJ/mol. The results of the docking model showed that: one carboxyl group in Asp5 was bound to ferrous ions with a distance to the ferrous ions of 2.6 Ä; guanidine nitrogen atoms in Arg11 was bound to ferrous ions with a distance of 2.1 Ä; one carboxyl group in Pro8 was bound to ferrous ions at a distance of 2.6 Ä. The above results showed that the two peptide segments GADGAPGKDGVRG (SEQ ID NO: 1) and GPRGDQGPVGR (SEQ ID NO: 2) had multiple binding sites for ferrous ions, and the ferrous ions were located in the middle of the polypeptide. This indicated that the two peptide segments showed a high binding activity to the ferrous ions.

TABLE 3
Peptide sequence identification of yak hide-derived oligopeptide ferrous
chelate

| Number | Peptide sequence | Mass | Score | Relative content |
|---|---|---|---|---|
| 1 | GADGAPGKDGVRG(SEQ ID NO: | 1155.56 | 108.70 | 16.23% |
| | 1) | 1094.56 | 205.09 | 11.32% |
| 2 | GPRGDQGPVGR (SEQ ID NO: 2) | | | |
| 3 | GPTGPIGSR (SEQ ID NO: 3) | 840.45 | 160.88 | 8.11% |
| 4 | GPAGPAGRP (SEQ ID NO: 4) | 778.41 | 170.94 | 6.33% |
| 5 | GRYY (SEQ ID NO: 5) | 557.26 | 83.13 | 6.26% |
| 6 | GEGGPQGPRG (SEQ ID NO: 6) | 910.43 | 136.1 | 5.01% |
| 7 | GPRGDQGPVGRS (SEQ ID NO: 7) | 1181.59 | 176.48 | 4.35% |
| 8 | FGFD (SEQ ID NO: 8) | 484.20 | 94.74 | 3.95% |
| 9 | FSGL (SEQ ID NO: 9) | 422.22 | 98.72 | 3.66% |
| 10 | GPAGPIGPV (SEQ ID NO: 10) | 763.42 | 161.77 | 2.90% |
| 11 | GPAGPQGPR (SEQ ID NO: 11) | 835.43 | 167.25 | 2.86% |
| 12 | LPQPPQE (SEQ ID NO: 12) | 807.41 | 119.07 | 2.67% |
| 13 | GPSGPAGKDGRIGQPG (SEQ ID | 1449.73 | 125.28 | 2.47% |
| | NO: 13) | | | |
| 14 | SGPAGPRGPPGSA (SEQ ID NO: | 1106.55 | 144.17 | 2.36% |
| | 14) | | | |
| 15 | GPVGPV (SEQ ID NO: 15) | 524.30 | 115.97 | 2.29% |
| 16 | GKSGDRGETGPAGPAGPIGPV | 1875.94 | 121.45 | 1.70% |
| | (SEQ ID NO: 16) | | | |
| 17 | PGEK (SEQ ID NO: 17) | 429.22 | 27.36 | 1.61% |
| 18 | SPDF (SEQ ID NO: 18) | 464.19 | 18.53 | 1.45% |
| 19 | GPAGKDGRIGQPG (SEQ ID NO: | 1208.63 | 119.68 | 1.26% |
| | 19) | | | |
| 20 | GERG (SEQ ID NO: 20) | 417.20 | 49.54 | 1.20% |
| 21 | GFDGDFY (SEQ ID NO: 21) | 819.31 | 148.37 | 1.12% |

### Example 7: Determination of Free Radical Scavenging Capacity of Yak Hide-Derived Oligopeptide Ferrous Chelate

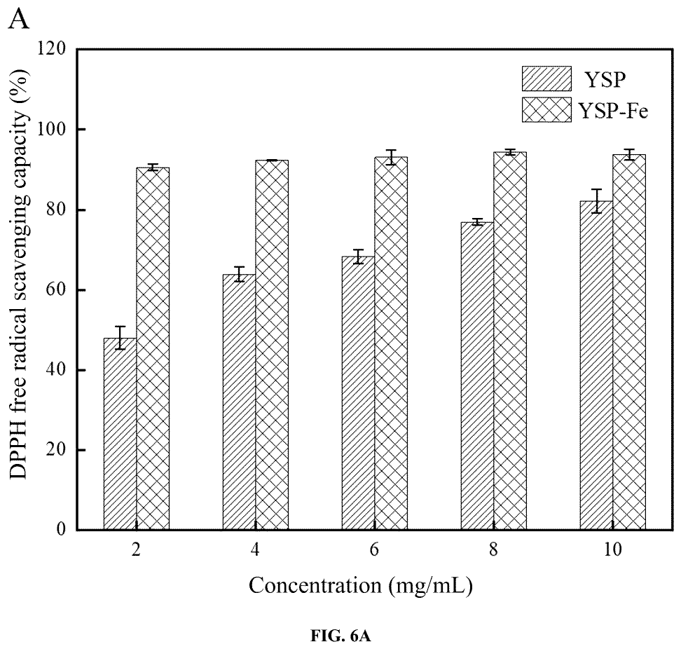

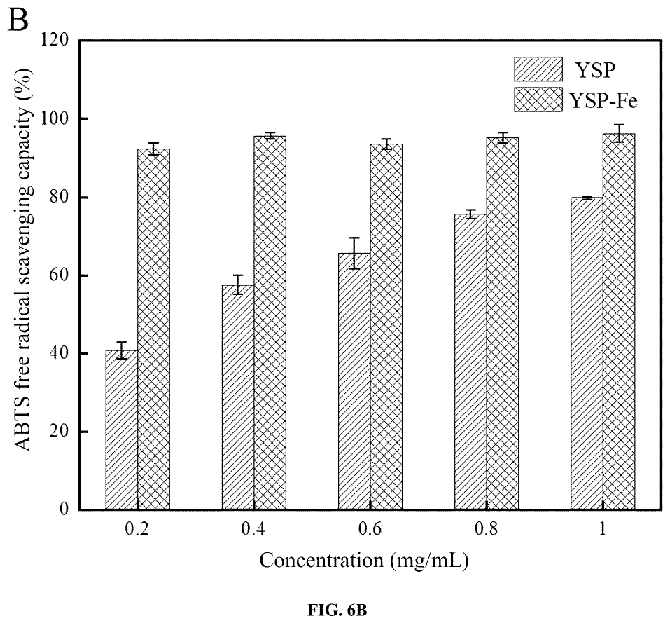

The different concentrations (2 mg/mL, 4 mg/mL, 6 mg/mL, 8 mg/mL, and 10 mg/mL) of yak hide-derived peptide/yak hide-derived iron-chelating peptide solutions were mixed in a volume ratio of 1:1 with 0.1 mmol/L DPPH ethanol solution. The absolute ethanol was then mixed with equal amounts of sample solution and 0.1 mmol/L DPPH absolute ethanol solution. All reactions were conducted in the dark for 30 min, and the absorbance was measured at 517 nm using a microplate spectrophotometer. As shown in FIG. 6A, the DPPH free radical scavenging capacity of the yak hide-derived iron-chelating peptide was significantly higher than that of the yak hide-derived peptide at the same concentration. The IC₅₀ values of the yak hide-derived oligopeptide and the yak hide-derived oligopeptide ferrous chelate for scavenging DPPH free radical were 2.22 mg/mL and 0.35 mg/mL, respectively.

An ABTS·⁺ solution containing 7 mmol/L ABTS solution and 2.45 mmol/L potassium persulfate solution was prepared. The ABTS·⁺ solution was allowed to stand in a light-proof and room-temperature environment for 12 h before use, and diluted with ethanol to make the absorbance value at 734 nm to 0.70±0.02 during use. 50 μL of yak hide-derived peptide and yak hide-derived iron-chelating peptide solutions of different concentrations (0.2 mg/mL, 0.4 mg/mL, 0.6 mg/mL, 0.8 mg/mL, and 1.0 mg/mL) were added with 200 μL of the diluted ABTS·⁺ solution separately, and then reacted in the dark at room temperature for 6 min, and the absorbance value was measured at 734 nm with a microplate spectrophotometer. As shown in FIG. 6B, the scavenging capacity of yak hide-derived iron-chelating peptide on ABTS free radical was significantly higher than that of yak hide-derived peptide at the same concentration. The IC₅₀ values of the yak hide-derived oligopeptide and the yak hide-derived oligopeptide ferrous chelate for scavenging ABTS free radical were 0.29 mg/mL and 0.03 mg/mL, respectively. Overall, the DPPH and ABTS free radical scavenging rates of yak hide-derived iron-chelating peptide were significantly lower than those of yak hide-derived peptide, indicating that the antioxidant properties of yak hide-derived iron-chelating peptide were higher than those of yak hide-derived peptide to a certain extent.

Based on the above analysis, yak hide-derived iron-chelating peptide had desirable free radical scavenging capacity and could be used as a potential antioxidant. In addition, chelation of metal ions might also produce antioxidant effects, as transition metal ions could promote oxidative damage to varying degrees.

At present, research by Athira et al. shows that after whey protein is chelated with iron, the antioxidant capacity of iron-chelating peptides is significantly lower than that of the original whey protein peptides. The IC₅₀ value of a sea cucumber-derived iron-chelating peptide prepared by Fan Chaozhong in scavenging ABTS free radicals is 1.40 mg/g, the IC₅₀ value of a walnut-derived iron-chelating peptide in scavenging DPPH free radicals is 3.72 mg/g, and the IC₅₀ values of the yak hide-derived iron-chelating peptide in the present disclosure in scavenging DPPH and ABTS free radicals are 0.35 mg/g and 0.03 mg/g, respectively. According to the above results, the IC₅₀ value of the yak hide-derived iron-chelating peptide prepared in the present disclosure in scavenging DPPH free radicals is significantly lower than that of walnut-derived iron-chelating peptide, and its IC₅₀ value in scavenging ABTS free radicals is significantly lower than that of sea cucumber-derived iron-chelating peptide. This indicates that the yak hide-derived iron-chelating peptide has a higher DPPH free radical scavenging capacity than that of walnut-derived iron-chelating peptide, and a higher ABTS free radical scavenging capacity than that of sea cucumber-derived iron-chelating peptide. To sum up, the yak hide-derived iron-chelating peptide is an iron-chelating peptide with a high antioxidant capacity.

Among the existing studies, use of yak hide-derived peptides in developing iron-chelating peptides has not been reported. Yak hide is generally discarded during production and processing, such that the present disclosure provides sufficient scientific basis for the development of novel iron supplements and high-value utilization of yak hide resources. In addition, the IC₅₀ value of yak hide-derived iron-chelating peptide in scavenging DPPH and ABTS free radicals is significantly lower than that of yak hide-derived peptide, indicating that the yak hide-derived oligocollagen peptide increases the antioxidant capacity after chelating with ferrous ions. Moreover, the antioxidant capacity of yak hide-derived oligomeric collagen peptide ferrous chelate is higher than that of most existing peptide-iron chelates, thus indicating that the yak hide-derived oligomeric collagen peptide ferrous chelate is a supplement with dual functions of iron supplementation and antioxidant.

Although the embodiments of the present disclosure have been disclosed for illustration, those skilled in the art shall understand that various replacements, changes and modifications are possible without departing from the spirit and scope of the present disclosure and the appended claims. Therefore, the scope of the present disclosure is not limited to what is disclosed in the embodiments and the accompanying drawings.

What is claimed is:

1. A preparation method of a yak hide-derived oligopeptide ferrous chelate with a high antioxidant activity, comprising subjecting a protein source and an iron source to chelation in water, wherein the protein source is a yak skin-derived collagen oligopeptide with a molecular weight of less than 2 kDa; and the yak skin-derived collagen oligopeptide and the iron source are subjected to the chelation in a mass ratio of 1:1 to 5:1 with a mass concentration of the yak skin-derived collagen oligopeptide of 1% to 5% under a pH value of 3 to 8 at 30° C. to 70° C. for 20 min to 60 min.

2. The preparation method according to claim 1, wherein the yak skin-derived collagen oligopeptide and the iron source are subjected to the chelation in a mass ratio of 2.7:1 with a mass concentration of the yak skin-derived collagen oligopeptide of 3% under a pH value of 6.8 at 50° C. for 40 min; and a corresponding prepared yak hide-derived oligopeptide ferrous chelate shows an iron chelating capacity of 42.72 mg/g+0.65 mg/g.

3. The preparation method according to claim 1, wherein the yak skin-derived collagen oligopeptide is a peptide segment with a relatively high iron chelating capacity and comprises SEQ ID NO: 1: GADGAPGKDGVRG and SEQ ID NO: 2: GPRGDQGPVGR.

4. The preparation method according to claim 1, specifically comprising the following steps:
(1) pretreating a yak hide: mechanically depilating the yak hide, removing subcutaneous muscle and fat, rinsing with water to remove surface soft flocks, cutting the yak hide into small pieces of 1 cm×1 cm, and subjecting the yak hide to defatting with a 5% Na₂CO₃ aqueous solution at 4° C. for 18 h to obtain a defatted yak hide; adding a NaCl solution with a mass fraction of 5% into the defatted yak hide and stirring continuously to remove salt-soluble non-collagen components, wherein the defatted yak hide and the NaCl solution are at a material-to-liquid ratio of 1:10, rinsing the defatted yak hide with distilled water multiple times to obtain a clean yak skin, and storing the clean yak hide at −20° C. for later use; adding the clean yak skin into 0.5 mol/L glacial acetic acid at a material-to-liquid ratio of 1:10 to allow swelling for 12 h, and conducting homogenization using a high-speed tissue masher at 10,000 r/min to obtain a yak hide homogenate to allow subsequent enzymatic hydrolysis;
(2) conducting enzymatic hydrolysis: subjecting the yak hide homogenate to enzymatic hydrolysis using different proteases of neutral protease, alkaline protease, flavor protease, complex protease, papain, and bromelain separately; wherein the enzymatic hydrolysis comprises: the enzymatic hydrolysis using the alkaline protease is conducted at a pH value of 10, while the enzymatic hydrolysis using the other proteases is conducted at a pH value of 7, and the enzymatic hydrolysis is conducted at 50° C. for 4 h with an amount of the protease added at 2% of a mass of the yak hide homogenate; heating a resulting enzymatic hydrolyzate at 95° C. for 15 min to terminate the enzymatic hydrolysis, and then conducting centrifugation under a room temperature at 5,000 r/min for 20 min to obtain a supernatant; precipitating a polysaccharide in the supernatant using absolute ethanol, and then conducting centrifugation at 4,000 r/min for 20 min to obtain a yak skin-derived collagen peptide solution obtained by the enzymatic hydrolysis using each of the different proteases;
(3) conducting separation and purification by gel chromatography: subjecting the yak skin-derived collagen peptide solution obtained by the enzymatic hydrolysis using each of the different proteases to separation and purification by gel chromatography to obtain a yak skin-derived collagen peptide; subjecting the yak skin-derived collagen peptide to enzymatic hydrolysis using the flavor protease under a pH value of 7 at 50° C. for 4 h with an amount of the flavor protease added at 2% of a mass of the yak skin-derived collagen peptide to obtain the yak hide-derived collagen oligopeptide;
(4) mixing the yak skin-derived collagen oligopeptide and a FeSO₄·7H₂O solution with a mass concentration of 1% to 5% to obtain a mixed solution, wherein the yak skin-derived collagen oligopeptide and the FeSO₄·7H₂O solution are at a mass-to-volume ratio of (0.1-0.5) g:10 mL, adjusting the mixed solution to a pH value of 3 to 8 with 1 mol/L NaOH or 1 mol/L HCl, and subjecting the mixed solution to the chelation at 30° C. to 70° C. for 20 min to 60 min; and
(5) adding 4 times a volume of the absolute ethanol into a resulting reaction product to precipitate a chelate of a yak hide-derived oligopeptide and ferrous ions after the chelation is completed; conducting centrifugation at 10,000 r/min for 15 min, and collecting a resulting precipitate to allow freeze-drying to obtain the yak hide-derived oligopeptide ferrous chelate with a high antioxidant activity.

5. The preparation method according to claim 1, wherein a yak hide-derived oligopeptide ferrous chelate prepared by the preparation method has a better capacity in scavenging a 2,2-diphenyl-1-picrylhydrazyl (DPPH) free radical and a 2,2′-azino-bis(3-ethylbenzothiazoline-6-sulfonic acid) (ABTS) free radical than that of a yak hide-derived oligomeric collagen peptide.

6. The preparation method according to claim 1, wherein the yak hide-derived oligopeptide ferrous chelate comprises 11.18%+1.08% of iron.

7. The preparation method according to claim 2, wherein the yak hide-derived oligopeptide ferrous chelate comprises 11.18%+1.08% of iron.

8. The preparation method according to claim 3, wherein the yak hide-derived oligopeptide ferrous chelate comprises 11.18%+1.08% of iron.

9. The preparation method according to claim 4, wherein the yak hide-derived oligopeptide ferrous chelate comprises 11.18%+1.08% of iron.

10. The preparation method according to claim 5, wherein the yak hide-derived oligopeptide ferrous chelate comprises 11.18%+1.08% of iron.

11. A yak hide-derived oligopeptide ferrous chelate prepared by the preparation method according to claim 1, wherein the yak hide-derived oligopeptide ferrous chelate is used for producing a food and/or a drug and/or a health care product.

12. The yak hide-derived oligopeptide ferrous chelate according to claim 11, wherein the yak skin-derived collagen oligopeptide and the iron source are subjected to the chelation in a mass ratio of 2.7:1 with a mass concentration of the yak skin-derived collagen oligopeptide of 3% under a pH value of 6.8 at 50° C. for 40 min; and a corresponding prepared yak hide-derived oligopeptide ferrous chelate shows an iron chelating capacity of 42.72 mg/g±0.65 mg/g.

13. The yak hide-derived oligopeptide ferrous chelate according to claim 11, wherein the yak skin-derived collagen oligopeptide is a peptide segment with a relatively high iron chelating capacity and comprises SEQ ID NO: 1: GADGAPGKDGVRG and SEQ ID NO: 2: GPRGDQGPVGR.

14. The yak hide-derived oligopeptide ferrous chelate according to claim 11, specifically comprising the following steps:
(1) pretreating a yak hide: mechanically depilating the yak hide, removing subcutaneous muscle and fat, rinsing with water to remove surface soft flocks, cutting the yak hide into small pieces of 1 cm×1 cm, and subjecting the yak hide to defatting with a 5% Na₂CO₃ aqueous solution at 4° C. for 18 h to obtain a defatted yak hide; adding a NaCl solution with a mass fraction of 5% into the defatted yak hide and stirring continuously to remove salt-soluble non-collagen components, wherein the defatted yak hide and the NaCl solution are at a material-to-liquid ratio of 1:10, rinsing the defatted yak hide with distilled water multiple times to obtain a clean yak skin, and storing the clean yak hide at −20° C. for later use; adding the clean yak skin into 0.5 mol/L glacial acetic acid at a material-to-liquid ratio of 1:10 to allow swelling for 12 h, and conducting homogenization using a high-speed tissue masher at 10,000 r/min to obtain a yak hide homogenate to allow subsequent enzymatic hydrolysis;
(2) conducting enzymatic hydrolysis: subjecting the yak hide homogenate to enzymatic hydrolysis using different proteases of neutral protease, alkaline protease, flavor protease, complex protease, papain, and bromelain separately; wherein the enzymatic hydrolysis comprises: the enzymatic hydrolysis using the alkaline protease is conducted at a pH value of 10, while the enzymatic hydrolysis using the other proteases is conducted at a pH value of 7, and the enzymatic hydrolysis is conducted at 50° C. for 4 h with an amount of the protease added at 2% of a mass of the yak hide homogenate; heating a resulting enzymatic hydrolyzate at 95° C. for 15 min to terminate the enzymatic hydrolysis, and then conducting centrifugation under a room temperature at 5,000 r/min for 20 min to obtain a supernatant; precipitating a polysaccharide in the supernatant using absolute ethanol, and then conducting centrifugation at 4,000 r/min for 20 min to obtain a yak skin-derived collagen peptide solution obtained by the enzymatic hydrolysis using each of the different proteases;
(3) conducting separation and purification by gel chromatography: subjecting the yak skin-derived collagen peptide solution obtained by the enzymatic hydrolysis using each of the different proteases to separation and purification by gel chromatography to obtain a yak skin-derived collagen peptide; subjecting the yak skin-derived collagen peptide to enzymatic hydrolysis using the flavor protease under a pH value of 7 at 50° C. for 4 h with an amount of the flavor protease added at 2% of a mass of the yak skin-derived collagen peptide to obtain the yak hide-derived collagen oligopeptide;
(4) mixing the yak skin-derived collagen oligopeptide and a FeSO₄·7H₂O solution with a mass concentration of 1% to 5% to obtain a mixed solution, wherein the yak skin-derived collagen oligopeptide and the FeSO₄·7H₂O solution are at a mass-to-volume ratio of (0.1-0.5) g:10 mL, adjusting the mixed solution to a pH value of 3 to 8 with 1 mol/L NaOH or 1 mol/L HCl, and subjecting the mixed solution to the chelation at 30° C. to 70° C. for 20 min to 60 min; and
(5) adding 4 times a volume of the absolute ethanol into a resulting reaction product to precipitate a chelate of a yak hide-derived oligopeptide and ferrous ions after the chelation is completed; conducting centrifugation at 10,000 r/min for 15 min, and collecting a resulting precipitate to allow freeze-drying to obtain the yak hide-derived oligopeptide ferrous chelate with a high antioxidant activity.

15. The yak hide-derived oligopeptide ferrous chelate according to claim 11, wherein a yak hide-derived oligopeptide ferrous chelate prepared by the preparation method has a better capacity in scavenging a 2,2-diphenyl-1-picrylhydrazyl (DPPH) free radical and a 2,2′-azino-bis(3-ethylbenzothiazoline-6-sulfonic acid) (ABTS) free radical than that of a yak hide-derived oligomeric collagen peptide.

16. The yak hide-derived oligopeptide ferrous chelate according to claim 11, wherein the yak hide-derived oligopeptide ferrous chelate comprises 11.18%±1.08% of iron.

17. The yak hide-derived oligopeptide ferrous chelate according to claim 12, wherein the yak hide-derived oligopeptide ferrous chelate comprises 11.18%±1.08% of iron.

18. The yak hide-derived oligopeptide ferrous chelate according to claim 13, wherein the yak hide-derived oligopeptide ferrous chelate comprises 11.18%±1.08% of iron.

19. The yak hide-derived oligopeptide ferrous chelate according to claim 14, wherein the yak hide-derived oligopeptide ferrous chelate comprises 11.18%±1.08% of iron.

20. The yak hide-derived oligopeptide ferrous chelate according to claim 15, wherein the yak hide-derived oligopeptide ferrous chelate comprises 11.18%±1.08% of iron.
